(19) United States
(12) Patent Application Publication
(10) Pub. No.: US 2011/0282376 A1
(43) Pub. Date: Nov. 17, 2011

(54) Brandow breast tunneler

(76) Inventor: Kirk Brandow, (US)

(21) Appl. No.: 12/800,382

(22) Filed: May 17, 2010

(51) Int. Cl. A61B 17/00

(52) U.S. Cl. 606/190

(57) ABSTRACT

An improved tunneling instrument for use in breast augmentation, having a distal end and a proximal end; includes a two piece system comprising of a penetrating bullet tip permanently attached to a hollow shaft and an attachable knurled handle. The shaft is constructed of stainless steel having sufficient rigidity. This tunneling instrument is designed, having multiple curves, which will allow the user to manipulate it in different directions while providing smooth contours during tunneling. The multiple curved design of this improved upon invention will allow the user to have full control during use; thereby preventing increased trauma to the surrounding tissues when tunneling. Due to the unique design of the current invention, the breast is given a more natural appearance after surgery.

### BACKGROUND OF INVENTION

1. Field of the Invention

This improved invention relates generally to surgical instruments, and more particularly to instruments known as tunneling devices used for subcutaneous access to a body cavity. This improved invention is comprised of a two piece curved tunneler specifically designed for use in breast augmentation.

2. Background of the Invention

It is commonly known that cosmetic breast surgery is a means to improve the appearance of a woman's breasts through implants or prosthesis which will alter the size and shape of the breast. Breast enhancement is referred to by several terms such as; breast augmentation, mammoplasty, breast enlargement, or even the slang term, boob job. Candidates for cosmetic breast surgery are usually women who desire larger breasts or who have sagging breasts, whose problem can be solved by breast enlargement. Tunneling is a required surgical step for transaxillary placement of breast implants, which is the most effective way to perform cosmetic breast surgery. The design of the tunneling instrument used in this procedure is an important key in minimizing trauma to the breast tissue and inferior border of the breast and to allow for a more positive healing response post-operatively.

Generally, there are two types of tunnelers: standard tunnelers and sheath tunnelers. Standard tunnelers draw a vascular graft through a dissected tissue tunnel which is created by insertion of a rigid, bullet tipped rod through a skin incision. One such example uses a two-part tunneler instrument which includes an oversized, relatively rigid metal or plastic hollow tube with a removable bullet shaped dissection tip on one end, and an internal smaller diameter indwelling rod for attaching the vascular graft material. Most rigid tunneling devices on the market today may at times be difficult to extract from the tissue due to the compressive tension on the surrounding tissue and surface friction thereby created. Tunneler instruments are generally used for placement of arteriovenous grafts for extracorporeal circulation of the blood, arterial bypasses, and the like. Another type of tunneler is the GORE sheath tunneler, which is produced by W. L. Gore and Associates, Inc of Flagstaff, Ariz. is a two part tunneler instrument used to implant a vascular graft subcutaneously with an oversized tissue passageway. The GORE tunneler is comprised of a hollow rigid metal shaft connected to a handle with a removable bullet tip at one end of the shaft. The shaft is fabricated from stainless steel and fits into a formed handle with a center rod. The instrument is used to bluntly dissect a tunnel by forcing the bullet tipped hollow shaft through the tissue. After suture attachment of the graft material to the inner rod the vascular graft is then easily drawn through the entire length of the oversized hollow tube. Another example of a two part rigid sheath tunneler is the “Scanlan” tunneler, which is similar in operative technique to the GORE device except that the rigid sheath is constructed of hard plastic and considered disposable (1 time use). Tunneling devices are expensive, and consequently hospitals maintain only a limited supply of these surgical devices for hospital use. Most instruments are reusable, and are required to be washed and sterilized between use.

This current invention is designed specifically for use in cosmetic breast surgery, but is an improvement from the tunneling devices on the market today. Tunneling instruments, while varying in size, can be difficult to control due to their design. Their designs often give poor control during use in breast surgery, resulting in a larger than required tunnel path, which may cause unnecessary and increased patient complications. During breast augmentation the tunneler must be manipulated with great care to insure precise placement of the implants during the surgical procedure.

The general object of this invention is to provide an improved tunneling instrument and method which will overcome the problems that one could anticipate if using the current designs on the market today. This improved invention will allow for simpler sterilization due to the permanently affixed bullet tip and removable knurled handle. Its curved design will allow the user to position the tunneler and manipulate it so that it will not create unnecessary bleeding and trauma to the surrounding area during surgery.

### BRIEF SUMMARY OF THE INVENTION

Broadly speaking, the present invention addresses the disadvantages of prior tunneling devices and provides for an easier better improved tunneling device which is designed to access the body cavity, specifically a woman's breasts for implants or prosthesis during cosmetic breast augmentation, using a blind keyhole method. According to this improved invention, the user can create a tunnel within a body cavity, while maintaining control and power during its use, thereby minimizing the chances of creating a much larger than required tunnel. The curved design creates a smooth tunnel around the inferior border of the breast using blunt dissection, yet minimizes trauma to the surrounding tissues specifically because of its design.

The tunneler of the current invention is a two piece device that includes a hollow shaft with a permanently attached bullet tip at the most distal end and a knurled handle at the most proximal end. The hollow shaft has a hole at the proximal end to be used for attaching it to the knurled handle. The knurled handle has a locking mechanism mounted into the distal portion of the handle; inside of a cavity disposed within. The benefit of the locking mechanism is that when it's compressed it will allow the handle to be attached to the shaft, locking it into place. Therefore, there is no need to thread the proximal end of the hollow shaft, nor the distal end of the knurled handle. The curves placed in the design of the present invention allows the user to manipulate the instrument around the natural curvature of the rib cage in order to place the implants correctly. The current tunnelers available are straight, such as the GORE tunneler, and are designed to insert grafts into a leg while in a straight position. They are generally rigid and bouncy and they have no curves as in the rib cage. The objects of the invention will become more apparent from the detailed description of the invention as follows.

### BRIEF DESCRIPTIONS OF THE DRAWINGS

The present improved upon invention will become better understood with reference to the following description and accompanying drawings, wherein:

FIG. 1 Is a depiction of an example of the hollow shaft portion of a curved tunneling instrument, comprised as a medical device used specifically for breast augmentation. Also shown is the bullet tip 15, which is permanently attached to the distal end of the hollow shaft. At the proximal end, there is a hole 13 in the hollow shaft for attaching the handle prior to use.

FIG. 1a Is an enlarged, broken away view of a depiction of a bullet tip 15 permanently attached to a tunneling instrument as shown on FIG. 1 of the current invention.

FIG. 1b Is a an enlarged, cross sectional view of the hole 13 which is shown at the proximal end of the hollow shaft FIG. 1 of the current invention; which is used for a locking mechanism as seen in FIGS. 2 and 2a. The locking mechanism FIG. 2a is used to attach the knurled handle FIG. 2 to the hollow shaft FIG. 1 of the tunneling instrument and will lock both the hollow shaft FIG. 1 and the knurled handle FIG. 2 into place giving both parts a flush fit when connected.

FIG. 2 Is a depiction of a further example of the knurled handle of the tunneling device of the current invention.

FIG. 2a Is an enlarged view of a depiction of a further example of the locking mechanism, which is shown in FIG. 2 at the distal end of the knurled handle, that when compressed, will allow the knurled handle FIG. 2 to lock into the hole at the proximal end of the hollow shaft FIG. 1. The locking mechanism is mounted into a cavity at the tip of the knurled handle.

FIG. 3 Is a representation of the positioning of a tunneling instrument within a patient's body for gaining transaxillary access to the breast through a blind keyhole 16 for the purpose of detaching the pectoralis muscle from the breast tissue for placement of implants or prosthesis. Also depicted are the placement of the knurled handle 14 in the users hand and the locking mechanism 13 which will not interfere with the manipulation of the tunneling device during use.

### DETAILED DESCRIPTION OF THE INVENTION

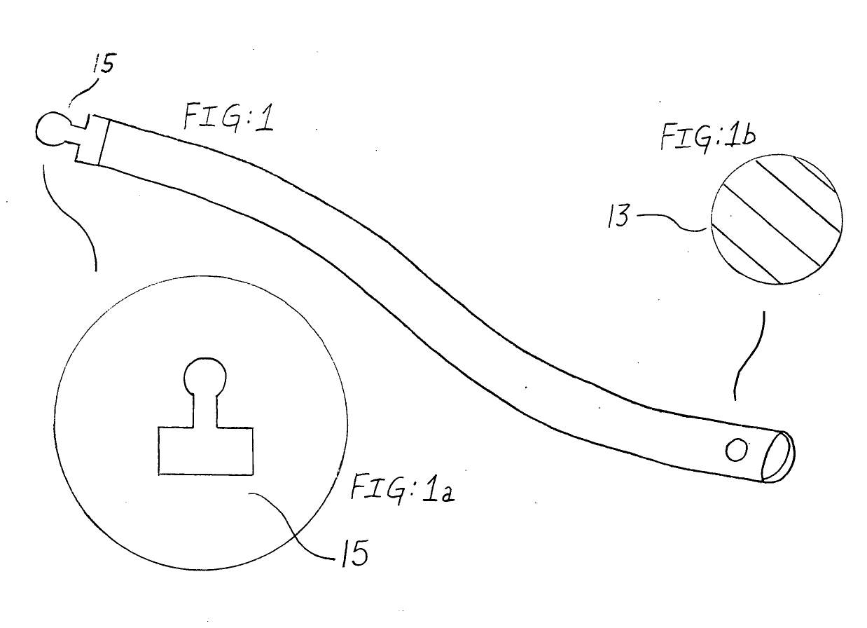

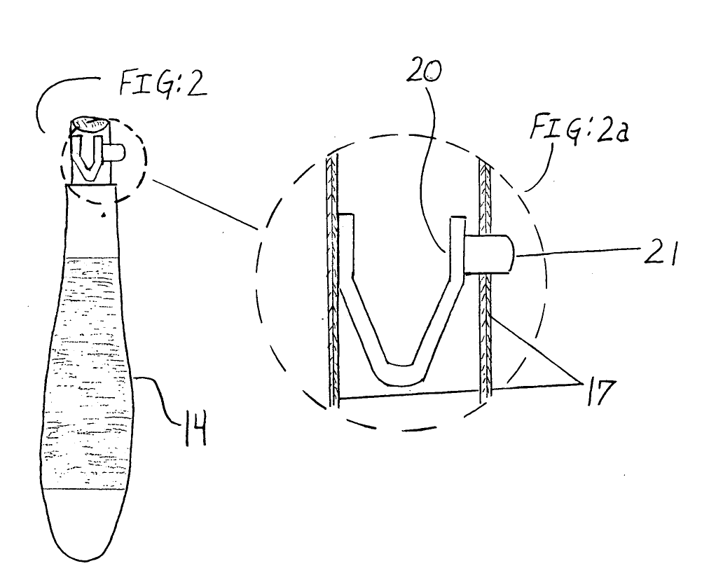

Referring now to the drawings wherein like referenced characters designate like or corresponding parts throughout the several views. FIG. 1 represents the hollow shaft portion of a tunneling instrument constructed according to the invention. The tunneler includes a hollow shaft FIG. 1, having a circular transverse cross section, with a bullet-like tip (15) at the distal end and a knurled handle FIG. 2 at the proximal end. The handle FIG. 2 is knurled in the center (14) which will allow the user maximum gripping of the tunneler when using it. The hollow shaft FIG. 1 is typically 6 to 8 mm. in diameter and is 1 mm thick, preferably made of a non-heat treated malleable metal, such as 300 series stainless steel, having sufficient stiffness.

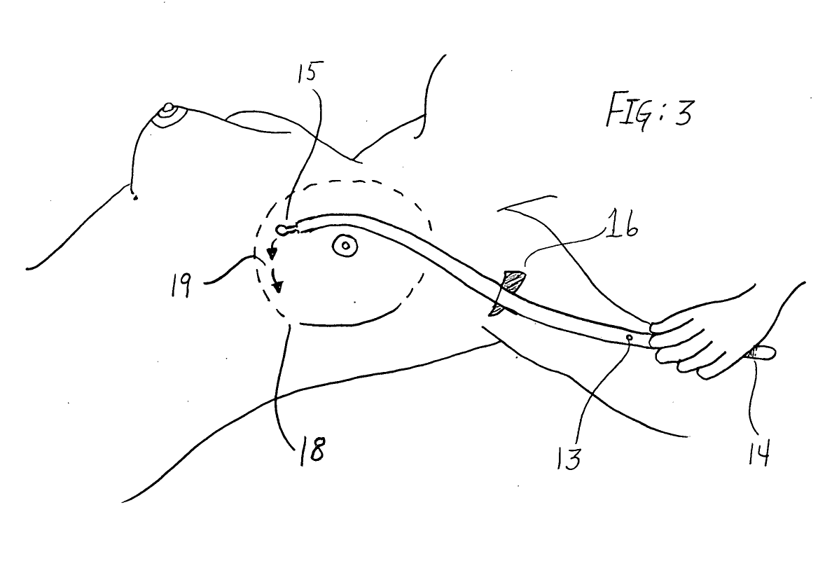

Tunneling instruments in the general case of medical devices are used for the purpose of harvesting the saphenous vein for bypass surgery and for implanting vascular grafts, such as natural tissue grafts, or similar implantable biological devices. There are many types of tunnelers available today, most of which are not appropriate for use in breast augmentation due to their poor control and design, which present clear disadvantages. This invention is not directed to a specific tunneling instrument, but is an improved tunneling instrument used specifically in breast augmentation procedures as seen on FIG. 3 of the drawings. This improved design comprises of a curved hollow shaft FIG. 1 with a bullet-like tip FIG. 15), which is permanently fixed at the distal end of the hollow shaft and a removable knurled handle FIG. 2 at the proximal end, having a locking mechanism FIG. 2a within a cavity at the tip of the distal end of the knurled handle.

Previously, large incisions had to be made when performing breast augmentation procedures, which resulted in longer surgery time and a longer healing period for the patient. With this improved tunneling instrument, surgery can be performed using only a 2 cm incision (16) and no retractors. Surgeons today, utilize multiple instruments to perform what can be a much simpler breast procedure when using the current improved upon invention. Dissectors while effective, tend to have a common defect, they do not allow the user full control and power to release the muscle from the breast tissue. Their less than caliber performance, results in longer healing time for the patient. Also, the current tunneling instruments in use today, tend to be too long and wobbly while others, due to their design do not give the user full control of use.

With this improved, curved tunneling instrument, the user will only require a 2 cm incision just under the axilla (16) to perform breast augmentation. The tunneler FIG. 1 is placed into a body cavity using the bullet tip (15) as a guide through a blind keyhole incision (16) for blunt dissection around the inferior border of the breast (18) as shown in FIG. 3, while the knurled handle FIG. 2 (14) is used to grip the tunneler and manipulate it during use. The curved design of this improved surgical tunneling instrument allows the surgeon to maintain good control and handling when detaching the muscle from the breast FIG. 3 (19) using a downward motion, while creating an accurate pocket size for the implant or prosthesis, giving the breast a more natural appearance post-operatively.

The accuracy from the use of this improved upon invention will minimize trauma to the surrounding tissues which, if occurs, can result in poor healing and is considered a major contributor of post-operative complications such as implant material infection, seroma formation or significant amounts of blood pooling around the entire border of the implant. This may cause implant failure due to poor healing of the localized tissue and painful swelling due to fluid accumulation between the dissected tissue and the implant. It is advantageous in the clinical setting to minimize trauma to this tissue through use of an improved tunneling and implant device such as the current invention. Unlike the current tunneling instruments on the market today which have removal tips, the bullet tip (15) of this current invention as seen in FIGS. 1, 1A and measuring about 2 cm is permanently attached to the hollow shaft FIG. 1 which will make autoclaving much easier. Current tips are typically removed so that they can be interchanged with other tips of different sizes, however this design has only one tip size as seen in FIG. 1A and does not require removal.

The knurled handle FIG. 2 measures about 9 cm in length with the distal tip (17) measuring about 2 cm in length and 5 mm in diameter. The locking mechanism FIG. 2a may be constructed of stainless steel, but one of ordinary skill in the art will recognize that other materials may be suitable. The locking mechanism FIG. 2a, wherein the protrusion is adapted to push to compress the elastic member (20) until the protrusion (21) clears and the knurled handle can be locked into the hollow shaft FIG. 1. The locking mechanism FIG. 2a is a one piece unit comprised of a flat piece of stainless steel, preferably and a protrusion (21) that is fitted within a cavity on the tip or most distal part of the knurled handle FIG. 2. When the locking mechanism FIG. 2a is compressed or pushed in, the knurled handle FIG. 2 will easily slide into the proximal end of the hollow shaft FIG. 1 locking it into place as to provide stabilization during use of the tunneler. The type of locking mechanism that is contemplated to be within the scope of this invention include, by way of example, the locking mechanism described in the following commonly assigned U.S. Pat. No. 7,322,371 B2 issued to Ching-Chuan You. The bullet tip (15) of the hollow shaft FIG. 1 measures about 2 cm in length. The knurled handle FIG. 2 is slightly tapered and is constructed of solid steel with exception of the tip (17) which has a cavity and which said cavity houses the locking mechanism FIG. 2a. The thickest part of the knurled handle FIG. 2 is at the proximal end and measures about 10 mm in diameter and 9 cm in length with tapering beginning about 4 cm from the proximal end of the handle FIG. 2 towards the distal part of the handle FIG. 2 and measures about 6 or 7 mm at the most distal part of the handle FIG. 2 just prior to the tip (17) wherein contains the locking mechanism; which when attached to the hollow shaft FIG. 1, will lock both parts together.

When the knurled handle FIG. 2 is connected to the hollow shaft FIG. 1, the entire length of this improved tunneling instrument is about 56 cm. The hollow shaft FIG. 1 which measures 47 cm in length, curves in one direction at about 8 cm from the proximal end and curves again in the other direction at about 16 cm and 24 cm from the proximal end. At about 35 cm from the proximal end, it begins curving once again in the opposite direction and continues to curve yet again at about 45 cm from the proximal end, but in the same direction of the curvature at 35 cm from the proximal end as shown in FIG. 1.

The general object of this invention is to provide a novel and improved tunneling instrument for use in cosmetic breast surgery which will overcome the aforementioned problems of the current tunneling devices on the market today. A further object of the invention is to provide an improved tunneling instrument for a more simplified method of use. This invention has been described and specific examples of the invention have been portrayed. While the invention has been described in terms of particular variations and illustrative figures, those of ordinary skill in the art, will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well. Finally, the patent application cited in this specification is herein incorporated by reference.

1. A tunneling instrument for providing access to the breast tissue through transaxillary approach using a blind keyhole technique.

2. The tunneling instrument of claim 1, wherein comprising of a hollow shaft that curves in opposite directions with the distal end, having a blunt tip for dissection of a subcutaneous tunnel within the breast wall.

3. The tunneling instrument of claims 1 and 2 having a permanently attached bullet tip for easier autoclaving.

4. The tunneling instrument of claim 1 having an attachable knurled handle, whereas the handle has a locking mechanism, that when compressed will securely attach the handle to the hollow shaft.

5. The tunneling instrument of claim 4, having an attachable handle, which comprises of a knurled affect in the center and a locking mechanism that is fitted into a cavity at the distal end and is permanently attached therein to prevent separation of connected parts during use of the tunneling instrument of claim 1.
